(19) United States
(12) Patent Application Publication
(10) Pub. No.: US 2004/0058909 A1
(43) Pub. Date: Mar. 25, 2004

(54) Method of treatment

(76) Inventor: Jeffrey Goldstein, Wilmington, DE (US)

Correspondence Address: ASTRA ZENECA PHARMACEUTICALS LP, GLOBAL INTELLECTUAL PROPERTY, 1800 CONCORD PIKE, WILMINGTON, DE, 19850-5437, US

(21) Appl. No.: 10/451,171

(22) PCT Filed: Dec. 18, 2001
(86) PCT No.: PCT/SE01/02820

(51) Int. Cl. A61K 31/554

(52) U.S. Cl. 514/211.13

(57) ABSTRACT

A method for treating dyskinesias associated with dopaminergic therapy is described using the a typical antipsychotic agent quetiapine. Also described is the co-administration of quetiapine and a dopaminergic agent for treating Parkinson's disease. Treatment with quetiapine suppresses the symptoms of Parkinson's disease and will attenuate levodopa-induced dyskinetic movements. This allows the dosage of dopaminergic agents, for example levodopa, to be increased without the complicating side-effects normally observed with higher doses.

This invention relates to a method for treating dyskinesias associated with dopaminergic therapy and in particular to the use of quetiapine in treating such disorders. The invention also relates to a method for treating Parkinson's disease by administration of quetiapine and a dopaminergic agent.

Quetiapine is an a typical antipsychotic agent which has good efficacy and tolerability and which is useful in the treatment of schizophrenia.

Dopaminergic therapy is a typical treatment for alleviating or partially alleviating the symptoms of Parkinson's Disease. Unfortunately, behavioural disorders and psychoses are complications of dopaminergic therapy and are difficult to treat.

Parkinson's Disease is a gradually progressive neurological disorder resulting from the loss of dopamine from that part of the brain responsible for co-ordination of motor movements, i.e. the basal ganglia. The main symptoms of Parkinson's Disease include tremor (shaking of the hands, feet or limbs), rigidity (stiffness, pain and cramps in muscles), bradykinesia (slowness of movement) and posture changes (difficulty in walking, maintaining balance, freezing). The cause of Parkinson's disease has yet to be determined, although it is not a hereditary disorder, and there is at present no cure for Parkinson's disease. The main treatment is drug therapy aimed at replacing the lost dopamine.

Quetiapine has been used in treating psychoses in patients suffering from Parkinson's Disease with no exacerbation of extra-pyramidal symptoms (Matheson and Lamb, Adis, CNS Drugs 2000, 14(2), 157-172). Matheson and Lamb conclude that quetiapine may be an effective alternative treatment to other antipsychotic agents without compromising motor function. Reference may also be made to two papers which describe the possibility of administering a typical antipsychotics as an adjunct to levodopa to treat the psychoses caused by levodopa therapy; Fernandez and Friedman: The role of a typical antipsychotics in the treatment of movement disorders, CNS Drugs 11(6):467483, 1999; and to Friedman and Factor: Atypical antipsychotics in the treatment of drug-induced psychosis in Parkinson's disease, Movement Disorders 15(2):201-211, 2000.

We have now found that quetiapine is useful in treating dyskinesias associated with dopaminergic therapy. Dyskinesias are restless, jerky, uncontrollable movements that can result from complications of dopaminergic therapy.

Therefore, according to the present invention, we provide a method for treating dyskinesias associated with dopaminergic therapy which comprises administering an effective amount of quetiapine or a pharmaceutically acceptable salt thereof to a patient in need thereof.

In another aspect, the present invention provides quetiapine or a pharmaceutically acceptable salt thereof for use in treating dyskinesias associated with dopaminergic therapy.

In yet a further aspect, the present invention provides the use of quetiapine or a pharmaceutically acceptable salt thereof in the manufacture of a medicament for treating dyskinesias associated with dopaminergic therapy.

By treating dyskinesias we include both reduction of dyskinesias and prophylactic treatment to prevent or ameliorate increased dyskinesias due to other therapy, for example dopaminergic therapy.

Conventional treatment of Parkinson's Disease includes treatment with levodopa and with related drugs such as dopaminergic agents. Levodopa and related drugs give rise to levodopa-induced dyskinesia; the familiar motor function problems that are observed in patients suffering from Parkinson's Disease. Treatment with quetiapine will suppress the symptoms of Parkinson's Disease and will attenuate levodopa-induced dyskinetic movements. This allows the dosage of dopaminergic agents, for example levodopa, to be increased without the complicating side-effects normally observed with higher doses.

Therefore, in another aspect the present invention provides a method for treating dyskinesias in a patient in need thereof which comprises co-administering to said patient a dopaminergic agent in an amount that together with quetiapine or a pharmaceutically acceptable salt thereof is effective to provide greater efficacy than provided by administering said dopaminergic agent alone.

Furthermore, treatment with quetiapine will enable a larger dosage of other drugs used to treat Parkinson's Disease or Parkinsonian symptoms. We further provide a method for treating dyskinesias associated with Parkinson's Disease which comprises co-administering an effective amount of quetiapine or a pharmaceutically acceptable salt thereof to a patient in need thereof together with a treatment for Parkinson's disease whereby the efficacy of the co-administered treatment is greater than that provided by the treatment for Parkinson's Disease alone.

In a further aspect we provide a method for treating Parkinson's Disease in a patient in need thereof which comprises concomitant administration of quetiapine or a pharmaceutically acceptable salt thereof and a dopaminergic agent; in particular where the dose of dopaminergic agent is greater than that administered to said patient in the absence of quetiapine or a pharmaceutically acceptable salt thereof. A particular benefit of such treatment is that the increased dose of dopaminergic agent enables the improved treatment of Parkinson's Disease and its symptoms without increased dyskinetic side-effects. Examples of dopaminergic agents include levodopa, bromocriptine, pergolide and amantadine. Preferably the dopaminergic agent is levodopa. Typically levodopa may be administered with a decarboxylase inhibitor for example carbidopa or benserazide; in particular levodopa and carbidopa may be administered concomitantly.

As mentioned above (Matheson and Lamb), quetiapine has been administered to psychotic patients who also suffer from Parkinson's Disease. The present invention further provides a method for treating dyskinesias associated with dopaminergic therapy which comprises administering an effective amount of quetiapine or a pharmaceutically acceptable salt thereof to a non-psychotic patient in need thereof. In yet a further aspect, the present invention further provides a method for treating dyskinesias associated with levadopa therapy which comprises administering an effective amount of quetiapine or a pharmaceutically acceptable salt thereof to a non-psychotic patient in need thereof. Non-psychotic patients typically do not display pathognomonic symptoms of psychosis for example delusions and hallucinations.

Quetiapine is 11-(4-[2-(2-hydroxyethoxy)ethyl]-l-piperazinyl)dibenzo[b,f] [1.4]-thiazepine. This compound, pharmaceutically acceptable salts thereof and its use in treating schizophrenia are described in granted European Patent No. EP 240,228 and in corresponding patents.

The method of treatment of the present invention may be conducted over a short term (5-6 weeks), medium term (1-6 months) or long term (6 months-2 years or more) treatment, and is particularly valuable in medium term and long term treatment. In a particular aspect, quetiapine does not exhibit the significant weight gain seen with some other a typical antipsychotics. Thus, it is particularly suitable for longer term treatment.

Quetiapine may be administered as the compound, 11-(4-[2-(2-hydroxyethoxy)ethyl]-1-piperazinyl)-dibenzo[b,f][1.4]thiazepine or may be administered in the form of a pharmaceutically acceptable salt. Examples of suitable salts include, for example, chloride, maleate, fumarate, citrate, phosphate, methane sulphonate and sulphate salts. Preferred salts include fumarates and a particularly preferred salt is the hemi-fumarate.

It is generally preferred that 11-(4-[2-(2-hydroxyethoxy)ethyl]-1-piperazinyl)-dibenzof[b,f] [1.4]thiazepine is administered in the form of a pharmaceutically acceptable salt, and in particular a fumarate (2:1) salt.

In the treatment of the diseases and conditions mentioned above quetiapine or a pharmaceutically acceptable salt may be administered orally or parenterally in a conventional dosage form such as tablets, pills, capsules, injectables or the like. The dosage in mg/kg of body weight of the compound used to treat mammals will vary according to the size of the mammal and particularly with respect to the brain/body weight ratio. In general, a higher mg/kg dosage for a small animal such as a dog will have the same effect as a lower mg/kg dosage in an adult human. A minimum effective dosage for quetiapine or a pharmaceutically acceptable salt thereof will be about 0.5 mg/kg of body weight per day for mammals with a maximum dosage for a small mammal such as a dog, of about 200 mg/kg per day.

For humans, a dosage of about 0.5 to 25 mg/kg per day will generally be effective. Typically, a dosage of about 50 mg to 1200 mg per day will generally be effective. Usually, a dosage of about 25 mg to about 1000 mg per day will be administered, with a convenient dosage being about 50-750 mg per day. In particular, a dosage of about 50-250 mg per day may be preferred such as about 75-150 mg per day. The dosage can be given once daily or in divided doses, for example, 2 to 4 doses daily. The dose may be conventionally formulated in an oral or parenteral dosage form by compounding 50 to 1200 mg per unit dosage of conventional vehicle, excipient, binder, preservative, stabiliser, flavour or the like as called for by accepted pharmaceutical practice, for example, as described in U.S. Pat. No. 3,755,340.

Quetiapine or a pharmaceutically acceptable salt may be used in pharmaceutical compositions as the sole active ingredient or may be contained in a pharmaceutical composition together with one or more other active ingredients, or it may be co-administered with one or more known drugs.

Dopaminergic agents typically may be administered according to methods known in the art for such agents, in dosage forms, at unit doses and at frequencies as determined by the skilled medical practitioner. For example levodopa typically may be administered orally from one to four times a day with a total daily dosage of 200 mg to 1200 mg dependent on the patient's condition, body weight and other factors.

As indicated above, where quetiapine or a pharmaceutically acceptable salt is administered in conjunction with another agent it may be co-administered simultaneously, sequentially or separately with that other agent or agents. Thus, as indicated above, quetiapine or a pharmaceutically acceptable salt may be formulated with the other agent or agents or may be presented as a separate formulation.

Thus, in one aspect of the present invention, there is provided a pharmaceutical composition comprising quetiapine or a pharmaceutically acceptable salt and a dopaminergic agent, for example levodopa, together with a pharmaceutically acceptable diluent or carrier.

In a further aspect there is provided a pharmaceutical composition comprising quetiapine or a pharmaceutically acceptable salt and a dopaminergic agent, for example levodopa, for simultaneous, sequential or separate administration.

In a yet further aspect of the present invention, there is provided a pharmaceutical composition comprising quetiapine or a pharmaceutically acceptable salt and an agent for treating Parkinson's Disease or Parkinsonian symptoms together with a pharmaceutically acceptable diluent or carrier.

In another aspect there is provided a pharmaceutical composition comprising quetiapine or a pharmaceutically acceptable salt and an agent for treating Parkinson's Disease or Parkinsonian symptoms for simultaneous, sequential or separate administration.

The preparation of 11-(4-[2-(2-hydroxyethoxy)ethyl]-1-piperazinyl)-dibenzo[b,f][1.4] thiazepine and its pharmaceutically acceptable salts is described in, for example, granted European Patents Nos. EP 240,218; EP 282,236 and in International Patent Application No. PCT/GB98/02260. This compound is commercially available under the generic name quetiapine fumarate.

The invention will now be illustrated with reference to the following, non-limiting examples in which quetiapine was used as the fumarate (2:1) salt.

### EXAMPLE 1

The benefit of quetiapine may be demonstrated as follows:

Quetiapine may be administered to cynomolgus monkeys exposed to 1-methyl-4-phenyl-1,2,3,6-tetrahydropyridine (MPTP) and which therefore have MPTP-induced Parkinson's Disease. Such monkeys are described by Blanchet et al., Experimental Neurology, 153, 214-222 (1998).

The monkeys are assessed according to the Laval University Disability Scale for MPTP monkeys with the following motor and behavioural parameters scored: posture (normal, flexed, crouched); mobility (normal, passive); climbing (present, absent); gait (normal, abnormal); tremor (present, absent); holding food (present, absent); vocalising (present, absent); grooming (present, absent); and social interaction (present, absent).

Quetiapine may be administered at various dosage values, either alone or with varying dosages of levodopa, and scoring is carried out at regular intervals.

Quetiapine was administered subcutaneously to 8 cynomolgus monkeys at doses of 2 mg/Kg and 4 mg/Kg. The monkeys had been treated with a dose of levodopa either sufficient to produce moderate dyskinesias or sufficient to produce maximum dyskinesias. Quetiapine at a dose of 4 mg/Kg reduced moderate levodopa-induced dyskinesias by 57% and reduced maximum levodopa-induced dyskinesias by 41%. Quetiapine at a dose of 2 mg/Kg was less effective. The levodopa effect on Parkinson's Disease symptoms was unchanged.

Quetiapine can reduce dyskinesia in the cynomolgus monkeys whilst the levodopa response is maintained.

### EXAMPLE 2

The following illustrates representative pharmaceutical dosage forms containing the compound 11-(4-[2-(2-hydroxyethoxy)ethyl]-1-piperazinyl)-dibenzo[b,f] [1,4] thiazepine fumarate (2:1).

| | mg/tablet |
|---|---|
| (a) Tablet | |
| Quetiapine fumarate | 50.0 |
| Mannitol, USP | 223.75 |
| Croscarmellose sodium | 6.0 |
| Maize starch | 15.0 |
| Hydroxypropylmethylcellulose (HPMC), | 2.25 |
| Magnesium stearate | 3.0 |
| (b) Capsule | |
| Quetiapine fumarate | 10.0 |
| Mannitol, USP | 488.5 |
| Croscarmellose sodium | 15.0 |
| Magnesium stearate | 1.5 |

The above formulations may be obtained by conventional procedures well known in the pharmaceutical art. The tablets may be enteric coated by conventional means, for example to provide a coating of cellulose acetate phthalate.

A preferred formulation is that available commercially as quetiapine fumarate.

### EXAMPLE 3

Levodopa is administered orally twice a day at a dosage of up to 2000 mg per day concomitantly with quetiapine fumarate (25 mg twice a day) to a patient in need thereof.

1. A method for treating dyskinesias associated with dopaminergic therapy which comprises administering an effective amount of quetiapine or a pharmaceutically acceptable salt thereof to a patient in need thereof.

2. A method for treating dyskinesias associated with levodopa therapy which comprises administering an effective amount of quetiapine or a pharmaceutically acceptable salt thereof to a patient in need thereof.

3. A method for treating dyskinesias associated with dopaminergic therapy, wherein the dopaminergic agent is administered to alleviate symptoms of Parkinson's Disease, which comprises administering an effective amount of quetiapine or a pharmaceutically acceptable salt thereof to a patient in need thereof.

4. A method for treating dyskinesias associated with levodopa therapy, wherein the dopaminergic agent is administered to alleviate symptoms of Parkinson's Disease, which comprises administering an effective amount of quetiapine or a pharmaceutically acceptable salt thereof to a patient in need thereof.

5. A method for treating dyskinesias associated with dopaminergic therapy which comprises administering an effective amount of quetiapine or a pharmaceutically acceptable salt thereof to a non-psychotic patient in need thereof.

6. Quetiapine or a pharmaceutically acceptable salt thereof for use in treating dyskinesias associated with dopaminergic therapy.

7. The use of quetiapine or a pharmaceutically acceptable salt thereof in the manufacture of a medicament for treating dyskinesias associated with dopaminergic therapy.

8. A method for treating dyskinesias in a patient in need thereof which comprises co-administering to said patient a dopaminergic agent in an amount that together with quetiapine or a pharmaceutically acceptable salt thereof is effective to provide greater efficacy than provided by administering said dopaminergic agent alone.

9. A method for treating dyskinesias in a patient in need thereof which comprises co-administering to said patient levodopa in an amount that together with quetiapine or a pharmaceutically acceptable salt thereof is effective to provide greater efficacy than provided by administering said levodopa alone.

10. A pharmaceutical composition which comprises a dopaminergic agent and quetiapine or a pharmaceutically acceptable salt thereof.

11. A method for treating Parkinson's Disease in a patient in need thereof which comprises concomitant administration of quetiapine or a pharmaceutically acceptable salt thereof and a dopaminergic agent.

12. A method according to claim 11 wherein the dose of dopaminergic agent is greater than that administered to said patient in the absence of quetiapine or a pharmaceutically acceptable salt thereof.

13. A method according to either claim 11 or 12 wherein the dopaminergic agent is levodopa.

14. A method according to claim 13 wherein the dopaminergic agent is levodopa and carbidopa.
